(19) United States
(12) Patent Application Publication
(10) Pub. No.: US 2026/0104416 A1
(43) Pub. Date: Apr. 16, 2026

(54) MULTIPLE BIOMARKERS FOR DIAGNOSING LUNG CANCER AND USE THEREOF

(71) Applicant: BeyondDX Inc., Gyeonggi-do (KR)

(72) Inventors: Je Yoel CHO, Seoul (KR); Hye Jin SUNG, Seoul (KR); Ji Young KIM, Seoul (KR)

(21) Appl. No.: 19/412,294

(22) Filed: Dec. 8, 2025

Related U.S. Application Data
(62) Division of application No. 17/799,529.

(30) Foreign Application Priority Data
Feb. 13, 2020 (KR) 10-2020-0017671

(51) Int. Cl. G01N 33/5752; C12Q 1/6876; G01N 33/575; G01N 33/68

(52) U.S. Cl. CPC G01N 33/5752; C12Q 1/6876; G01N 33/5758; G01N 33/68

(57) ABSTRACT

The present invention relates to a composition for diagnosing lung cancer including a preparation capable of measuring expression levels of lung cancer-specific biomarkers SAA, OPN, and CEA at the same time, a kit for diagnosing lung cancer including the same, and a method of diagnosing lung cancer using the composition. The composition for diagnosing lung cancer has effects such as enhanced sensitivity and specificity as compared to conventional biomarkers, thereby exhibiting high diagnostic efficiency.

### CROSS-REFERENCE TO RELATED APPLICATIONS

This is a divisional application under 35 USC § 120 of U.S. patent application Ser. No. 17/799,529 filed Aug. 12, 2022, which in turn is a U.S. national phase under 35 USC § 371 of International Patent Application PCT/KR2021/001790 filed Feb. 10, 2021, which claims priority under 35 USC § 119 to Republic of Korea Patent Application 10-2020-0017671 filed Feb. 13, 2020. All such applications are hereby incorporated by reference herein, in their respective entireties, for all purposes.

### TECHNICAL FIELD

The present invention relates to a multiple biomarkers for diagnosing lung cancer including a plurality of lung cancer-specific biomarkers, and a method of diagnosing lung cancer using the same.

### BACKGROUND ART

In Korea, lung cancer ranks first in cancer deaths and accounts for one in every five cancer deaths (approximately 22.8%). The recent data from the National Statistical Office in Korea showed that the mortality rates of gastric cancer, liver cancer, and the like have tended to decrease, but the mortality rate of lung cancer has tended to increase by approximately 20% since ten years ago. Despite the striking advances in modern medicine, the incidence and mortality rate of lung cancer are increasing continuously, and most patients find that they have lung cancer at the end phase because they have no subjective symptoms at the initial phase of lung cancer, which makes it difficult to apply various therapies to lung cancer after diagnosis of the disease. Therefore, when lung cancer is found early at a period of time when the disease is treatable through screening before its symptoms are expressed, it is expected to improve the survival rate and reduce the mortality rate.

In recent years, methods mainly used in screening through which lung cancer can be diagnosed include X-rays, CT, MRI, and the like. However, all of these methods have drawbacks in that it is difficult to diagnose lung cancer at an early stage, its screening method is cumbersome, and patients may be exposed to radiation during periodic physical examinations. Also, because most patients undergo examinations after their cancer is already progressed significantly, the prognosis is not good even after treatment. Therefore, to solve these problems, there is an urgent need for the development of a novel method of diagnosing lung cancer, which is simple and has a high diagnostic rate.

A biomarker is a substance that is measured as an objective indicator of normal biological processes, pathological processes, or a pharmacological response to treatment, and thus is closely related to the cause and progression of a certain disease. A method of diagnosing the disease using the biomarker has an advantage in that the disease may be found at an early stage by only analyzing a patient's specimen such as blood, or the like. Therefore, it is inevitable to develop a biomarker for diagnosing lung cancer, which is difficult to detect at an early stage using conventional diagnosis methods. Also, it is expected that the developed biomarker will be able to play an important role in diagnosis and targeted treatment of cancer. Several single biomarkers for diagnosing lung cancer are currently being used in clinical trials, but have problems in that they have low specificity and sensitivity, resulting in a low diagnostic rate. Because the biomarkers' specificity and sensitivity values continuously vary depending on the individual's genetic traits and living environments, in particular, because they are closely associated with various factors such as cancer incidence and cancer microenvironments, it is, in fact, impossible to accurately diagnose lung cancer using only a single biomarker. Accordingly, there is a need for the development of novel multiple biomarkers capable of improving specificity and sensitivity in order to diagnose lung cancer with high accuracy and sensitivity.

### DISCLOSURE

### Technical Problem

A technical objective of the present invention is to provide a composition for diagnosing lung cancer, which includes a preparation for measuring an expression level of mRNAs of serum amyloid A (SAA), osteopontin (OPN) and carcinoembryonic antigen (CEA) genes, or proteins thereof, and a method of providing information for diagnosis of lung cancer using the same.

### Technical Solution

In order to solve the above problems, a composition for diagnosing lung cancer according to the present invention comprises: a preparation for measuring an expression level of mRNAs of serum amyloid A (SAA), osteopontin (OPN) and carcinoembryonic antigen (CEA) genes, or proteins thereof.

That is, the present invention relates to a composition for diagnosing lung cancer capable of detecting and diagnosing an expression level of the serum amyloid A (SAA), osteopontin (OPN) and carcinoembryonic antigen (CEA) proteins or genes encoding the proteins at the same time so that the proteins can be used as a biomarker for diagnosing lung cancer as the same time.

In this specification, the term “diagnosis” refers to a process of identifying the onset and characteristics of a target disease. According to one aspect of the present invention, the diagnosis is for identifying the onset and degree of progression (that is, stage) of lung cancer.

In this specification, the term “marker” refers to a substance that can distinguish and diagnose a normal subject and a lung cancer subject. Here, the marker includes organic biomolecules such as polypeptides, proteins, or nucleic acids, genes, lipids, glycolipids, glycoproteins, and the like, which show an increase or decrease in expression level in a subject having lung cancer according to the present invention. Particularly, in this specification. SAA. OPN and CEA, which have expression levels that vary in a biological specimen taken from a subject having lung cancer, were used as a biomarker.

In the present invention, the expression “preparation for measuring an expression level of mRNAs or proteins thereof” refers to a molecule that can be used to detect and quantify a biomarker by checking an mRNA or protein expression level of serum amyloid A (SAA), osteopontin (OPN) and carcinoembryonic antigen (CEA) which are biomarkers whose expression levels vary in a lung cancer patient. Specifically, a preparation for measuring an mRNA expression level of the genes may be a primer pair, a probe, or an anti-sense nucleotide that specifically binds to the genes. Also, a preparation for measuring an expression level of the proteins may be an antibody that specifically binds to the proteins or fragments including amino acid sequences derived from the proteins. In this case, the type of antibody is not limited.

In this specification, the term “primer pair” includes all combinations of primer pairs consisting of forward and reverse primers, both of which recognize a target gene sequence. Specifically, the primer pair is a primer pair that provides analysis results with specificity and sensitivity. Because a nucleic acid sequence of a primer is a sequence which is not matched with an off-target sequence present in a specimen, the primer may impart high specificity when it is a primer that is used to amplify only a target gene sequence containing a complementary primer binding site without causing non-specific amplifications.

In this specification, the term “probe” refers to a substance that may specifically bind to a target material to be detected in a specimen, and also refers to a substance that may specifically determine whether there is a target material in a specimen by means of the binding. Probe molecule types include materials commonly used in the art, but the present invention is not limited thereto. Preferably, the probe may be a peptide nucleic acid (PNA), a locked nucleic acid (LNA), a peptide, a polypeptide, a protein. RNA, or DNA. More specifically, the probe is a biomaterial, and includes those derived from living organisms or synthesized in vitro, for example, enzymes, proteins, antibodies, microorganisms, animal and plant cells and organs, neurons. DNA, and RNA. In this case. DNA may include cDNA, genomic DNA, oligonucleotides. RNA may include genomic RNA. mRNA, oligonucleotides, and examples of the proteins may include antibodies, antigens, enzymes, peptides, and the like.

In this specification, the term “anti-sense” sequence refers to DNA or RNA, or a derivative thereof, which contains a nucleic acid sequence complementary to a sequence of certain mRNA, and acts to inhibit translation of mRNA into a protein by binding to a complementary sequence in the mRNA. An anti-sense oligonucleotide sequence refers to a DNA or RNA sequence that is complementary to mRNAs of the genes and can bind to the mRNAs. This may inhibit essential activities for the translation of genomic mRNA, translocation into the cytoplasm, maturation, or other overall biological functions.

In this specification, the term “antibody.” refers to a protein molecule that specifically binds to an antigenic site. For the purpose of the present invention, the antibody refers to an antibody that specifically binds to a marker protein, and includes all types of polyclonal antibodies, monoclonal antibodies, recombinant antibodies, and the like. The antibody may be easily prepared using techniques widely known in the related art. Also, the antibody used in this specification includes an intact form of an antibody having two full-length light chains and two full-length heavy chains, as well as functional fragments of the antibody molecule. The functional fragments of the antibody molecule refer to fragments that retain at least an antigen-binding function, and include Fab, F(ab′), F(ab′) 2, Fv, and the like.

According to one aspect of the present invention, the present invention provides a kit for diagnosing lung cancer including the composition. The details of the composition are as described above, and the kit may include a preparation for measuring an expression level of mRNAs of SAA, OPN and CEA genes, or proteins thereof, for example, a primer pair, a probe, or an anti-sense nucleotide, which specifically binds to the genes, and may also include antibodies, or antigen-binding fragments, which specifically bind to the marker proteins.

Also, the present invention provides a method of providing information for diagnosis of lung cancer, which includes measuring expression levels of mRNAs or proteins of serum amyloid A (SAA), ostcopontin (OPN) and carcinoembryonic antigen (CEA) genes from a biological specimen isolated from a subject suspected of having lung cancer; and comparing an expression level of the proteins encoded by the genes, or the protein-derived fragments with that of the normal control.

According to one aspect of the present invention, the method of providing information includes measuring an expression level of mRNAs or proteins of serum amyloid A (SAA), osteopontin (OPN) and carcinoembryonic antigen (CEA) genes. The expression level of the mRNAs may be measured using a method such as a reverse transcription-polymerase chain reaction (RT-PCR), a competitive reverse transcription-polymerase chain reaction (competitive RT-PCR), a real-time quantitative reverse transcription-polymerase chain reaction (real-time quantitative RT-PCR), a quantitative reverse transcription-polymerase chain reaction (quantitative RT PCR), an RNase protection assay, Northern blotting, or a DNA chip array, and the expression level of the proteins may be measured using a method selected from Western blotting, an immunohistochemical staining assay, an immunoprecipitation assay, a complement fixation assay, an immunofluorescence test, a radioimmunosorbent test, or mass spectrometry, but the present invention is not limited thereto.

The method of providing information for diagnosis of lung cancer according to the present invention includes comparing the measured expression level of the mRNAs or proteins with that of the normal control. In this case, the onset of lung cancer or the severity of the disease may be determined depending on a degree of change in expression level of mRNAs or proteins of marker genes in a specimen from a subject (i.e., a patient) suspected of having lung cancer.

### Advantageous Effects

The present invention provides a composition for diagnosing lung cancer including a preparation capable of measuring expression levels of lung cancer-specific biomarkers SAA, OPN, and CEA at the same time, a kit including the same, and a method of diagnosing lung cancer using the composition. In this case, the diagnostic composition can have enhanced effects such as sensitivity and specificity as compared to conventional biomarkers, thereby exhibiting high diagnostic efficiency.

### DESCRIPTION OF DRAWINGS

FIG. 1 shows the Box Plot results of comparing expression levels of SAA, OPN and CEA proteins from biological specimens of a normal person and a lung cancer patient;

FIG. 2 shows the results of determining abilities to diagnose lung cancer using each of the SAA, OPN and CEA proteins in the biological specimen of the lung cancer patient as a single biomarker, which are indicated as a receiver operating characteristic (ROC) curve:

FIG. 3 shows the results of determining abilities to diagnose lung cancer using a combination of contents of the SAA, OPN and CEA proteins in the biological specimen of the lung cancer patient as the biomarker, which are indicated using Box Plot and an ROC curve:

FIG. 4 shows the results of determining abilities to diagnose lung cancer in the lung cancer patient with individual stages using combined proteins according to the present invention, which are indicated by Box Plot:

FIG. 5 shows the results of determining abilities to diagnose lung cancer in the lung cancer patient with individual stages using the combined proteins according to the present invention, which are indicated as an ROC curve:

FIG. 6 shows the results of determining diagnostic performance for proteins in the lung cancer patient's serum using the multiple biomarkers of the present invention, which are determined using an ROC curve and an area under the ROC curve (AUC) value; and

FIG. 7 shows the results of determining diagnostic performance of the single biomarkers for diagnosing lung cancer, which are determined using an ROC curve and an AUC value.

### MODE FOR INVENTION

Hereinafter, the present invention will be described in detail.

In this specification, the term “serum amyloid A” (hereinafter referred to as “SAA”) is known as a precursor of an inflammatory condition of reactive amyloidosis, and thus refers to a main acute-phase protein whose serum concentration increases in various body conditions. The SAA multigene family includes four similar genes. It is known that, among proteins encoded by the genes, SAA1 and SAA2 are main proteins that appear in the acute-phase response, SAA3 is a pseudogene, and SAA4 is constitutively expressed. According to one embodiment of the present invention, an expression level of the SAA1 protein in a patient's blood was determined using an antibody against the protein as a target.

In this specification, the term “osteopontin” (hereinafter referred to as “OPN”) is a protein that is commonly found in interstitial fluid, and is expressed in activated T cells or plasmacytoid dendritic cells (DCs), which is regulated by a transcription factor (i.e., T-bet). Osteopontin was first known to be a non-collagenous bone matrix protein, since then, it has been known to play an important role in the regulation of cytokine secretion or cellular migration in the immune system, and the like. In particular, osteopontin is a factor that is very critical in differentiating CD4 T cells into TH1 in an immune response, and is also known to play an important role in the regulation of the macrophage activity and the migration of neutrophils into a site of inflammation.

In this specification, the term “carcinoembryonic antigen” (hereinafter referred to as “CEA”) refers to one example of a carcinoembryonic protein that is a tumor antigen in a prenatal period, and is often used as a tumor indicator. CEA is a substance that originally is normally present in the fetal intestinal tissue, but is secreted into blood when cancer develops in adults. CEA increases due to colon cancer, pancreatic cancer, liver diseases or various benign diseases, smoking or alcohol drinking, and the like.

In this specification, the expression “biomarker for diagnosing lung cancer” refers to an organic biomolecular material that may be used to diagnose lung cancer because its expression level increases or decreases in cells, tissues, or the like from lung cancer, compared to normal cells. In the present invention, all of SAA, OPN and CEA are used as biomarkers, and their expression levels may be determined at an mRNA or protein level to identify the onset and stage of lung cancer. The biomarkers of the present invention show both improved specificity and sensitivity compared to the single biomarker, and have a characteristic of very high diagnostic efficiency for lung cancer.

As will be seen from one embodiment of the present invention, the multiple biomarkers of the present invention are selected by combining multiple biomarkers having an excellent diagnostic ability from a number of single biomarkers using a T-test. According to one embodiment of the present invention, performance of the combination of multiple biomarkers is assessed through regression analysis. The diagnostic performance of the composition for diagnosing lung cancer, which is used to detect the multiple biomarkers of the present invention, is determined according to the following equation, which indicates that the multiple biomarkers have a very high diagnostic ability, compared to when the lung cancer is diagnosed using the respective single biomarkers.

$$x = α + {β}_{1} \times ( SAA Con . ) + {β}_{2} \times ( OPN Con . ) + {β}_{3} \times ( CEA Con . ) \qquad Equation l$$

wherein α is in a range of −50≤α≤10, β₁ is in a range of −5≤β₁≤20, β₂ is in a range of −5≤β₂≤10, and β₃ is in a range of −10≤β₃≤5.

The quantitative analysis data of the three proteins used in the present invention is used to select the multiple combined biomarkers having a considerably improved diagnostic ability compared to the performance as the single biomarkers, and the regression analysis model of Equation 1 is used to maximize an effect of a combination of the multiple biomarkers using the optimum weighted value for a variable for each biomarker. Based on the analysis as described above, the multiple biomarkers of a combination of SAA, OPN and CEA according to the present invention may be used as markers having an excellent ability to diagnose lung cancer.

Hereinafter, the present invention will be described in detail with reference to embodiments thereof in order to describe this specification in detail. However, it should be understood that the embodiments according to this specification may be modified into various other forms, and are not construed to limit the scope of this specification. The embodiments of this specification are provided to those with ordinary skill in the art in order to describe the present invention more completely.

### Experimental Example 1: Collection of Blood Samples of Experimental Group and Control

Serum samples of lung cancer patients (1,129) and normal persons (700) were provided from Seoul National University Bundang Hospital, Samsung Medical Center, and Kyungpook National University Hospital, and analyzed. The lung cancer patients ranged from 20 to 85 years old (65 years on average), the normal persons ranged from 21 to 87 years old (51 years on average), and the lung cancer patients were distributed by stages: stages 1 and 2:371, stage 3:253, and stage 4:316. The serum samples were obtained by taking peripheral blood from the lung cancer patients and the normal persons, storing the peripheral blood at room temperature for an hour, and centrifuging the peripheral blood to obtain a supernatant. The supernatant was stored at −80° C. until use.

### Experimental Example 2: Separation of Proteins in Serum

A concentration of each of three protein biomarkers (SAA, OPN, and CEA) in the serum collected in Experimental Example 1 was measured using an ELISA method. The SAA and OPN proteins were measured using a Duoset ELISA kit from R&D System, and the CEA protein was measured using an antibody from Biospecific Inc. The analyses were performed in a 96-well plate according to the respective protocols.

Wells of the 96-well plate were coated overnight with capture antibodies for SAA, OPN, and CEA at 4° C., and then a standard material or a serum were added into each well, and reacted for an hour. Thereafter, the reaction solution was treated with a biotin-tagged detector antibody, and reacted for an hour. Then, a streptavidin-horseradish peroxidase conjugate was added thereto, and the resulting mixture was reacted at room temperature for 30 minutes. 3,3′,5,5′-tetramethylbenzidine (TMB) was added to induce a chromogenic reaction. After 15 minutes, the reaction was stopped with sulfuric acid, and the absorbance at 450 nm was measured using a microplate reader.

The results of absorbance measurement were analyzed by 5-parameter curve fitting. The information on the ELISA kit, the antibodies, and the standard material used in this Example are listed in Table 1 below.

TABLE 1

| Biomarker | Product Name | Manufacturer |
|---|---|---|
| SAA | Human Serum Amyloid A1 DuoSet | R&D Systems |
| | ELISA | |
| OPN | Human Osteopontin (OPN) DuoSet | R&D Systems |
| | ELISA | |
| CEA | CEA monoclonal antibody | Biospecific |
| | | Inc. |
| | CEA protein | Fitzgerald |

### Experimental Example 3: Selection of Biomarkers for Diagnosing Lung Cancer

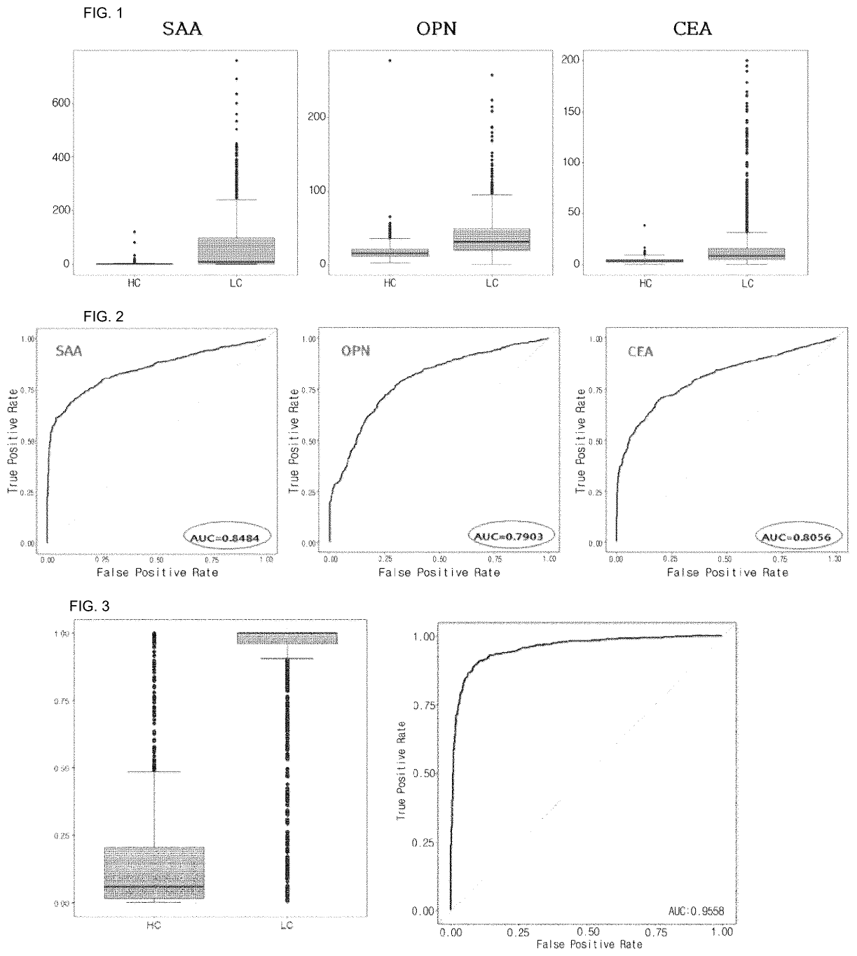

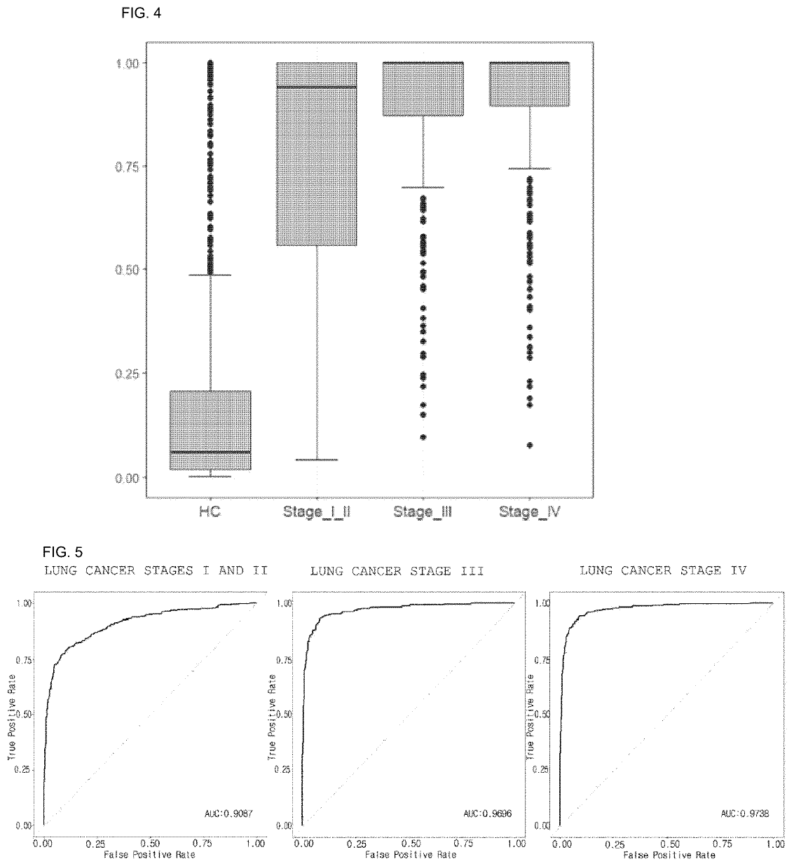

Biomarkers capable of being usefully used for diagnosis of lung cancer by specifically showing a change in values in the lung cancer patients' blood were screened. Based on the results analyzed in Experimental Examples 1 and 2, it can be seen that the expression levels of the SAA, OPN, and CEA proteins were remarkably high in the group of lung cancer patients, compared to the group of normal persons (FIG. 1)

Each of the identified biomarkers was assessed for an ability to diagnose lung cancer. Based on the sensitivity and specificity measured to diagnose actual lung cancer patients with lung cancer, each of the biomarkers was actually checked for having a diagnostic ability via the ROC curve. As a result, it can be seen that the AUC values were 0.8484, 0.7903, and 0.8056 for the SAA, OPN, and CEA biomarkers, respectively (FIG. 2).

### Experimental Example 4: Evaluation of Ability of Biomarker Combination to Diagnose Lung Cancer

The three biomarkers screened through the T-test were combined to assess a diagnostic ability for lung cancer using regression analysis. An algorithm for performance evaluation uses the following equation.

$$x = α + {β}_{1} \times ( SAA Con . ) + {β}_{2} \times ( OPN Con . ) + {β}_{3} \times ( CEA Con . ) \qquad Equation l$$

wherein α is in a range of −50≤α≤10, Bi is in a range of −5≤β₁≤20, β₂ is in a range of −5≤β₂≤10, and β₃ is in a range of −10≤β₃≤5.

The algorithm is an equation for calculating an estimated value (X) of a probability of being diagnosed with lung cancer, and was used as an index value to calculate estimated values (X) of probability of being likely to be lung cancer compared to the normal conditions and classify the respective estimated values (X) into normal and lung cancer categories. The estimated values (X) are in a range of −Δ∞8 values. In this case, the closer to −∞8, the more likely to be diagnosed with a normal condition and the closer to ∞8, the more likely to be diagnosed with lung cancer. β₁, β₂, and β₃ are regression coefficients representing weights for concentration values of the SAA, OPN, and CEA proteins, respectively, and α represents an intercept value when the estimated value (X) is 0.

The diagnostic ability of the combination of three biomarkers according to the present invention was assessed using the algorithm. As a result, it was confirmed that the combination of biomarkers showed excellent diagnostic performance with a specificity of 90%, a sensitivity of 90% or more, and an AUC of 0.9558 (see Table 2 and FIG. 3 below).

TABLE 2

| | | | | | | |
|---|---|---|---|---|---|---|
| Sensitivity | 99% | 98% | 95% | 90% | 85% | 80% |
| Specificity | 58% | 70% | 84% | 90% | 92% | 94% |

Also, the lung cancer patients in the experimental group were classified by stage (stages 1 and 2, stage 3, and stage 4), and then their ROC curves were analyzed using the same combination of biomarkers. As a result, it can be seen that the combination of biomarkers had a high AUC value in the terminal lung cancer patients as well as the early (stages 1 and 2) lung cancer patients, that is, that the combination of biomarkers had both high sensitivity and specificity (FIG. 5).

TABLE 3

| Specificity | | 99% | 98% | 95% | 90% | 85% | 80% |
|---|---|---|---|---|---|---|---|
| Sensitivity | Stages 1 and 2 | 45% | 57% | 71% | 80% | 82% | 83% |
| | Stage 3 | 69% | 76% | 89% | 94% | 95% | 96% |
| | Stage 4 | 68% | 79% | 90% | 94% | 97% | 97% |

### Experimental Example 5: Confirmation of Clinical Significance of Diagnostic Kit Using Multiple Biomarkers

A kit for diagnosing lung cancer (PROTAN LC-Check FL) was manufactured from the identified ability of the combination of biomarkers to diagnose lung cancer. To determine whether a combination of SAA, OPN, and CEA markers in the developed kit shows statistical significance between lung cancer patients and normal persons, proteins in the patients' sera were isolated in the same manner as in Experimental Example 2, and analyzed using bioinformatics and a statistical analysis method (i.e., R statistical package). The statistical analysis was performed using a logistic regression model, and the performance of prediction results for verification of the combination of biomarkers was confirmed by an AUC value of the ROC curve. This was used to deduce an algorithm for indicating a risk of lung cancer, and this algorithm was used to evaluate diagnostic performance for lung cancer.

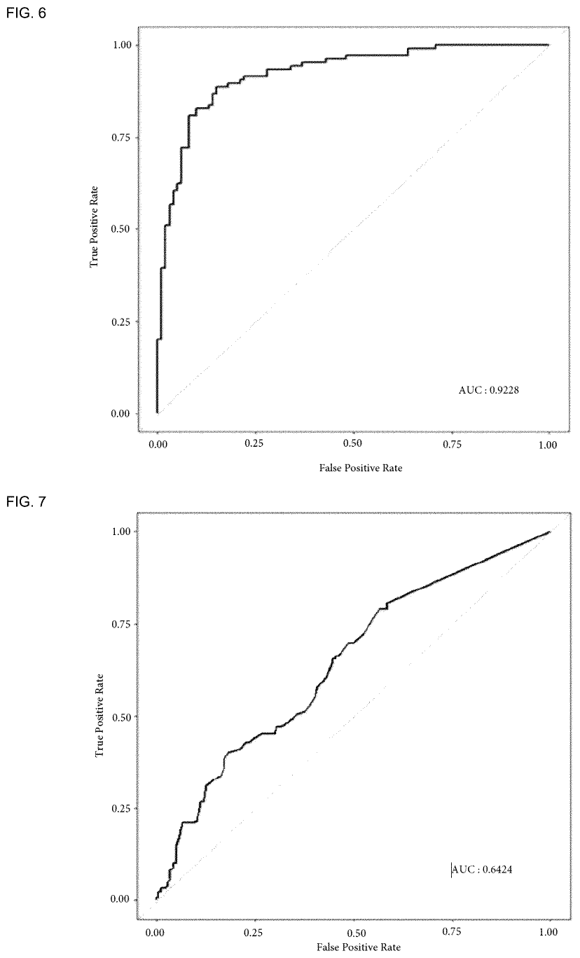

As a result, as shown in Table 4 and FIG. 6 below, it was confirmed that the diagnostic kit of the present invention had excellent performance with a specificity of 85%, a sensitivity of 87%, and an AUC of 0.9228.

TABLE 4

| | | | | | | |
|---|---|---|---|---|---|---|
| Sensitivity | 99% | 98% | 95% | 90% | 85% | 80% |
| Specificity | 17% | 39% | 61% | 81% | 87% | 89% |

Also, the same experiment was performed using a commercially available single biomarker (Cyfra21-1) to compare the diagnostic performance of the diagnostic kit according to the present invention. As a result, it was confirmed that the AUC of the Cyfra21-1 was measured to be 0.6424, indicating that the diagnostic kit of the present invention showed significantly superior performance (FIG. 7).

Until now, the present invention has been shown and described with reference to preferred embodiments thereof. It will be understood by those skilled in the art to which the present invention pertains that various changes in form and details may be made therein without departing from the spirit and scope of the present invention. Therefore, the preferred embodiments of the present invention should be considered in a descriptive sense only and not for purposes of limitation. Accordingly, it is clear that the scope of the present invention is defined not by the detailed description of the present invention but by the appended claims, and all differences within an equivalent range should be construed as being included in the present invention.

What is claimed is:

1. A method of providing information for diagnosis of lung cancer, comprising:
screening a biological specimen from a subject suspected of having lung cancer;
measuring expression levels of serum amyloid A (SAA), osteopontin (OPN) and carcinoembryonic antigen (CEA) genes, or proteins encoded by the genes or protein-derived fragments, from the specimen; and
comparing the expression level of the genes, or the proteins encoded by the genes or the protein-derived fragments with that of the normal control.

2. The method according to claim 1, wherein the expression level of mRNAs is measured using a method selected from a reverse transcription-polymerase chain reaction (RT-PCR), a competitive reverse transcription-polymerase chain reaction (competitive RT-PCR), a real-time quantitative reverse transcription-polymerase chain reaction (real-time quantitative RT-PCR), a quantitative reverse transcription-polymerase chain reaction (quantitative RT-PCR), an RNase protection assay, Northern blotting, or a DNA chip array.

3. The method according to claim 1, wherein the expression level of the proteins or protein-derived fragments is measured using a method selected from an enzyme-linked immunosorbent assay, Western blotting, an immunohistochemical staining assay, an immunoprecipitation assay, a complement fixation assay, an immunofluorescence test, a radioimmunosorbent test, or mass spectrometry.

4. The method of claim 1, wherein comparing the expression level of the genes for providing information for diagnosis of lung cancer comprises a computer-implemented statistical analysis of the expression level of the specimen using a logistic regression model.

5. The method of claim 4, wherein the logistic regression model comprises an algorithm for determining an estimated value of a probability of being diagnosed with lung cancer.

6. The method of claim 5, wherein the algorithm comprises an equation for calculation of the estimated value x of a probability of being diagnosed with lung cancer, wherein the equation comprises
$$x = α + {β}_{1} \times ( SAA Con . ) + {β}_{2} \times ( OPN Con . ) + {β}_{3} \times ( CEA Con . )$$
wherein:
α is an intercept value when x=0;
SAA Con. is a concentration of SAA expression in the specimen;
OPN Con. is a concentration of OPN expression in the specimen;
CEA Con. is a concentration of CEA expression in the specimen;
β₁, β₂, and β₃ are regression coefficients representing weights for concentrations of SAA expression, OPN expression, and CEA expression, respectively.
